SEQUENCE LISTING (1 sequence)

SEQ ID NO: 1 - protein, 394 aa, Artificial Sequence
  Synthesized
EDPQGDAAQKTDTSHHDQDHPTFNKITPNLAEFAFSLYRQLAHQSNSTNILFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAVFLEAIPVSIPPEVKFNKPFVFLMIDQNTKSPLFMGKVVNPTQK

(19) United States
(12) Patent Application Publication
(10) Pub. No.: US 2020/0354433 A1
(43) Pub. Date: Nov. 12, 2020

(54) RECOMBINANT MUTANT (alpha)1-ANTITRYPSIN AND PREPARATION AND USES THEREOF

(71) Applicant: PEKING UNIVERSITY, Beijing (CN)

(72) Inventors: Xinli LIN, PITTSBURG, CA (US); Lanfen LI, BEIJING (CN)

(21) Appl. No.: 16/941,658

(22) Filed: Jul. 29, 2020

Related U.S. Application Data
(63) Continuation of application No. PCT/CN2018/105011, filed on Sep. 11, 2018.

(30) Foreign Application Priority Data
Jan. 31, 2018 (CN) 201810093705.0

(51) Int. Cl. C07K 14/81; C12N 15/63; C07K 1/20

(52) U.S. Cl. CPC C07K 14/8125; A61K 38/00; C07K 1/20; C12N 15/63

(57) ABSTRACT

Provided is a new AAT triple-mutant, and methods to produce and purify the new entity. The new mutant is produced by a structure-based protein design to provide a more thermostable and oxidation-resistant agent for various pharmaceutical applications. The present invention also provides methods for E. coli expression, inclusion body refolding, and purification of the triple-mutant. Furthermore, the invention also provides methods for chemically modifying the purified drug candidate to provide a longer in vivo half-life and achieve better drug efficacy.

### CROSS-REFERENCE TO RELATED APPLICATIONS

This application is a continuation of International Patent Application No. PCT/CN2018/105011 with a filing date of Sep. 11, 2018, designating the United States, now pending, and further claims priority to Chinese Patent Application No. 201810093705.0 with a filing date of Jan. 31, 2018. The content of the aforementioned applications, including any intervening amendments thereto, are incorporated herein by reference.

### TECHNICAL FIELD

This disclosure relates to a recombinant mutant al-Antitrypsin (AAT) polypeptide and its preparation and uses in the medical field.

### BACKGROUND

Alpha-1 antitrypsin (AAT) was discovered as early as 1963 to be linked to hereditary emphysema. In the 1970s, researchers discovered the function of AAT and established AAT deficiency (AATD) as a risk factor for emphysema. Physiologically, neutrophils that can pass through the pulmonary capillaries freely are important mediators in the immune response for microbial pathogens invading lung cells. Neutrophils that served as part of an inflammatory response can also release a large number of defensive molecules, including reactive molecule oxygen, cationic peptides, arachidonic acids, and proteolytic enzymes. Even though these pathogen-killer molecules are essential part of the defensive system, they could also cause severe lung damage if out of control.

Previous research has established that a serine protease, human leukocyte elastase (HLE), is released from the azurophilic granules of the neutrophil as part of the normal inflammatory response. Under normal homeostatic conditions, AAT serves as an important regulator of proteolysis by HLE to prevent damage to the lung alveolar matrix. AAT is a 52 kDa glycoprotein synthesized primarily in the liver but can also be made in neutrophils, monocytes, and macrophages. AAT is secreted into the blood plasma, but its primary site of action is in the lung parenchyma. Besides HLE, AAT also inhibits two other proteases released into the lungs by neutrophils, namely cathepsin G (CatG) and proteinase 3 (Pr3). CatG and Pr3 may also contribute to lung damage by breaking down elastin and other extracellular matrix proteins. By inhibiting the function of these two proteases, AAT may, in turn, prevent their potential damage even though HLE is considered to be the enzyme primarily responsible for lung damage. To sum up, the normal biological function of AAT is essential for human health, and has been called ‘a guardian of vascular tissue’.

It is estimated that there are 253,404 AAT null mutation population (Pi*ZZ) worldwide: 119,594 in Europe, 91,490 in America and the Caribbean, 3,824 in Africa, 32,154 in Asia, 4,126 in Australia, and 2,216 in New Zealand. These mutant carriers suffer from hereditary emphysema and may be treated with AAT augmentation therapy. Globally it is estimated that about 3 million deaths were caused by chronic obstructive pulmonary disease (COPD) in 2015, according to the World Health Organization, and in many cases can be treated with AAT therapy. However, the AAT drugs which are currently on the market (Prolastin®, Aralast®, Zemaira®, Glassia®) are all derived from human serum and can satisfy less than 10% of the AAT-deficient population only. Because of the limited supply of human serum and, therefore, native AAT, it has not been adequately tested for its beneficial effects in other respiratory disorders, which include emphysema caused by smoking, cystic fibrosis, pulmonary hypertension, pulmonary fibrosis, and COPD. Worldwide, it is estimated that there are at least 116 million alpha one carriers (PiMS and PiMZ: M, normal genetic Pi Type; S, lower than normal; Z, lower than S) and 3.4 million with deficiency allele combinations (PiSS, PiSZ, and PiZZ), all of whom are more sensitive to environmental conditions such as smoke and air pollution, and more easily to develop COPD.

In order to manufacture recombinant AAT for replacing the native drug in the market, many different expression systems have been used. These include bacteria, yeast, plant cultures, and transgenic sheep. Because of the large dose required for hereditary emphysema (4-6 g·week-1 per patient injectable), and up to 250 mg daily for aerosolized lung delivery, the essential requirements for manufacturing AAT are scalability—mega kilos of pharmaceutical-grade drug required per year—and cost control—to make the drug affordable to patients. E. coli expression is one of the most cost-effective production methods for recombinant protein drugs. However, AAT tends to form insoluble inclusion bodies when overexpressed in bacteria, limiting the scalability of soluble expression.

### SUMMARY

Native α1-antitrypsin (AAT) is a 52-KDa glycoprotein that acts as an antiprotease, and is the physiological inhibitor of serine proteases neutrophil elastase, cathepsin G, and protease 3. The primary function of AAT is to protect the lung from proteolytic damage induced by inflammation. The genetic or acquired deficiency of AAT increases the risk of pulmonary emphysema and chronic obstructive pulmonary disease (COPD). Determined by its biological function, native AAT is relatively unstable and easily be oxidized. In order to make a better drug for clinical application, the thermostability, sensitivity to oxidation, and in vivo half-life of the native AAT need to be improved.

In this invention, using the efficient inclusion refolding technologies developed in our laboratory, we refolded and purified recombinant AAT from inclusion bodies. In addition, to meet the requirement of the clinical treatment, we developed a more thermostable and oxidation resistant mutant AAT to overcome some shortcomings of the wild type AAT, such as sensitive to oxidation and reduced stability of non-glycosylated protein produced in E. coli. In this invention, we designed and screened several mutant forms of AAT, and successfully obtained a new oxidation-resistant and thermostable triple mutant, which is more suitable for clinical application. Furthermore, to prolong/extend the half-life of AAT in vivo, we designed, produced, and purified a chemically modified AAT at specific site. The novel chemically modified mutant in this invention made manufacturing recombinant AAT protein drug in E. coli possible on a large scale.

We have expressed AAT in Escherichia coli as inclusion bodies and developed a highly efficient refolding and purification method. We engineered a series of mutant forms of AAT to achieve enhancement of thermostability and oxidation resistance. Moreover, we synthesized an active form of AAT via cysteine-pegylation and fatty acid modification to achieve a markedly extended in vivo half-life. The resulting molecule is expected to be an improved therapeutic agent for treating hereditary emphysema. Besides, the molecule may also be used to treat other types of emphysema caused by smoking, cystic fibrosis, pulmonary hypertension, pulmonary fibrosis, and chronic obstructive pulmonary disease (COPD).

The goal of this invention is to develop a recombinant AAT therapeutics by using an efficient and cost-effective expression system to generate rationally designed muteins (mutant proteins) for both increased thermostability and oxidation resistance. Also, we were able to make site-specific chemical modifications to the protein, which is expected to result in a longer in vivo half-life. The E. coli expression and refolding system we developed can achieve high yields of highly pure protein at a relatively low cost. This invention can solve some significant problems for therapeutic applications of the recombinant wild-type protein. The first problem is that the wild-type AAT has a tendency to be oxidized, and is unstable under physiological conditions. The present invention attempts to solve this problem by constructing and selecting more stable and oxidation-resistant mutant proteins, making it more suitable for therapeutic development. The second problem is that the AAT expressed in E. coli is non-glycosylated, making the in vivo half-life much shorter than the native, glycosylated one. Therefore, the present invention has designed chemical modifications of the E. coli expressed recombinant AAT, including PEGylation and fatty acid modification (such as Palmitoylation), to improve the stability and in vivo half-life. The mechanism of PEGylation is increasing the size of therapeutic proteins to reduce in vivo clearance, blocking the recognition of proteins by the immune system, and prevention of the degradation of therapeutic protein drugs by proteases. The mechanism of Palmitoylation is that the in vivo half-life would be prolonged by binding to human serum albumin after systemic administrated. The binding increases the in vivo stability and decreases in vivo clearance of therapeutic peptides or proteins.

In order to obtain a better therapeutic recombinant AAT protein drug and a more stable rAAT, we designed multiple mutants based on the AAT crystal structure, one of which was F51L mutant protein. As shown in FIG. 7, the wild-type phenylalanine residue at position 51 is buried in the hydrophobic core of the molecule, far away from the active site. After a round of nonspecific chemical mutagenesis and selection, Kwon et al. demonstrated that aliphatic amino acid substitutions at this position confer dramatically increased thermal stability of AAT without prompting inactivation, aggregation, or a change of association kinetics with the elastase. The non-glycosylated E. coli-expressed mutant was proved to slow down heat inactivation more than 10-fold at 57° C., such that the mutant behaved like plasma-derived glycosylated AAT. In this study, we replaced this residue with a leucine (F51L) after modeling with a crystal structure (FIG. 7). Results have shown that this substitution confers dramatically increased thermal stability of AAT (FIG. 5). It is important to know that the thermal stability of AAT has been proved to be related to the biological turnover rate of the protein. Therefore, better thermal stability of AAT is more optimal for drug development.

The second designed mutation in this invention is oxidation resistance. It is well known that AAT is very sensitive to oxidation due to its in vivo functional regulation requirements. Some known components in inhaled cigarette smoke, such as hydrogen peroxide, can easily oxidize AAT in the lung. Furthermore, it has been hypothesized that one of the pathophysiological causes of the smokers' lung disease is the decrease of active AAT in the lungs. The most susceptible residues are Met351 and Met358, which happen to be in the P8 and P1 positions of the AAT binding site (FIG. 7), respectively. Conservative substitution of each of these positions with another aliphatic amino acid such as valine resulted in a molecule that was considerably resistant to oxidation by hydrogen peroxide and slightly affected AAT's association kinetics or binding to target enzyme neutrophil elastase. Our research has shown that the double mutant-M351V/M358V confers a substantial increase in oxidation resistance (FIG. 6).

Our goal is to construct a novel combinatorial mutant F51L/M351V/M358V to achieve both stability and antioxidant performance. As is known to those skilled in the art in this field that any change in even one amino acid of the protein may lead to the instability and alter the nature of the protein; that's why the expression level, stability, and purification conditions of any new mutants, especially multi-mutants, can be unpredictable. In addition, it can be seen from the above early research papers concerning single and double mutants of AAT that both remain in the stage of purely theoretical research due to the limitation of expression and purification technology. Our study found that the expression level of full-length AAT was low in both single, double, and triple mutants. The yield could be used for laboratory research, but not for the application of new therapeutic development.

Through many tests and innovative exploration, we eventually constructed a novel combination of stabilization and oxidation-resistant triple mutant, F51L/M351V/M358V, which is fully active and shows both enhanced thermostability and resistance to oxidation (FIGS. 5 and 6). Under the expression conditions and renaturation methods of this invention, both the natural protein and the mutant (we find that the expression and renaturation conditions of the mutant and the natural protein are different) have reached unprecedented expression levels. In terms of application, it is a process not only from quantitative to qualitative change but also from purely theoretical research to the development and preparation of an applicable recombinant new drug.

The in vivo half-life of the non-glycosylated AAT produced in E. coli is much shorter than the native, glycosylated AAT. For example, the plasma half-life of glycosylated rat AAT is 170 minutes in rat serum, while it is only 30 min for the non-glycosylated form of the same molecule. One of the most effective methods for extending the in vivo half-life and reducing the immune reaction of a protein is polyethylene glycol conjugation (PEGylation). As shown in FIG. 4, we have successfully pegylated Cys232 of the wild-type AAT according to the published procedure. Cys232 is the only cysteine in AAT and exists as a monomeric molecule (FIG. 7). Therefore, Cys232 is accessible to a site-specific PEGylation. The pegylated material behaved like the wild-type AAT in inhibiting PPE (FIG. 4) in vitro, with similar association rates.

In summary, we have expressed recombinant AAT in E. coli with high yields in inclusion body form, which has been purified and refolded successfully, and developed highly efficient refolding and purification procedures for pharmaceutical drug development. In addition, we produced not only a novel triple-mutant form of AAT with enhanced thermal stability and oxidation resistance but also a pegylated form, further extending the in vivo half-life. The resulting entity could potentially have superior druggable properties for a range of medical applications than the native AAT from human serum.

It should be noted that references cited by this invention represent all references that can be retrieved. The AAT polypeptide and AAT protein described in this invention have equivalent meanings. In some descriptions, AAT represents both the wild type and the mutant form of the invention.

This invention provides a novel al-antitrypsin (AAT) mutant and a method for producing and purifying this mutein. The novel mutein was discovered by structure based design for a more suitable, structurally stable, and antioxidant candidate protein drugs for therapeutic applications. This invention also provides a method for expression from Escherichia coli, renaturation of the resulting inclusion bodies, and purification of the new mutein. Furthermore, this invention also provides a method for chemical modification of the purified candidate drug to extend the half-life of the protein drug in vivo and achieve better therapeutic efficacy.

### BRIEF DESCRIPTION OF THE DRAWINGS

FIG. 1. Protein sequence of the mature AAT and the amino acid substitution positions of the designed mutants. The starting position of Δ5AAT is shown. An additional starting Met was installed in the Escherichia coli expression vector. Positions of amino acid changes are labeled with underlines. The mutant numbers in the text are based on the mature, full-length AAT. The nucleotide changes of three mutants are listed as follows: F51L , TTT→CTG ; M351V/M358V, ATG→GTG/ATG→GTG; and F51L/M351V/M358V, TTT→CTG/ATG→GTG/ATG→GTG.

FIG. 2. SDS-PAGE of expressed wild-type, F51L, M351V/M358V, and F51L/M351V/M358V muteins. Proteins were stained by Coomassie Brilliant Blue. A small-scale expression was performed. Lanes 1-3: purified wild-type AAT inclusion bodies were loaded 1 μl, 3 μl, 5 μl, respectively. Lane 4: BSA marker. Lane 5: MW standard. Lanes 6-8: Purified F51L mutein inclusion bodies were loaded 5 μl, 3 μl, 1 μl, respectively. Lanes 9-19: expression test of soluble and insoluble cell extract. Lanes 9, 11: wild-type, soluble extract; lanes 10, 12: wildtype, insoluble extract. Lanes 13, 15: M351V/M358V, soluble extract; lanes 14, 16: M351V/M358V, insoluble extract. Lane 17: MW standard. Lane 18: F51L/M351V/M358V, soluble extract. Lane 19: F51L/M351V/M358V, insoluble extract.

FIGS. 3A-C. SDS-PAGE of purified recombinant AAT and muteins (A), and stoichiometric inhibition of PPE (B, C). (A) Purified samples were concentrated and applied to the SDS-PAGE and stained with Coomassie Brilliant Blue. Lane 1: wild-type AAT; lanes 2-4: increasing quantities were loaded to check purity (1 μg, 2 μg, 4 μg, respectively); Lane 2: F51L; Lane 3: M351V/M358V; Lane 4: F51L/M351V/M358V. Molecular weight markers (MW) indicated. (B) The activity of recombinant AAT (WT) compared with Zemaira®, a commercial native AAT drug working as a reference. (C) The activity of purified muteins, as compared to the WT.

FIGS. 4A-D. Cys232 PEGylation, purification, and properties. (A) Cation exchange column profile of pegylated AAT. (B) Nonreduced SDS-PAGE of fractions from the Q XL column. Serial numbers corresponding to gradient fractions were shown in (A). (C) MALDI-TOF mass spectrometry of a sample after PEGylation reaction. The molecular weight of each of the indicated peaks is depicted at the top of the respective peak. (D) The inhibitory activity of purified, pegylated AAT in blocking PPE.

FIG. 5. Comparison of thermal stability of wild-type and mutant AAT. The figure shows the counts of fluorescence (y-axis) against the temperature (x-axis, ° C.).

FIG. 6. Oxidation-resistant assay. The generation of aminolysis activity was monitored (at 405 nm) at 37° C. in 10 s intervals for 20 min using SpectraMax 250 microplate reader (Molecular Devices). The IC50 (y-axis) of each H202-treated AAT or its mutants were determined using GRAFIT version 7 (Erithacus Software, erithacus.com). The x-axis shows the molar ratio of H202 and AAT (H2O2:AAT, from 4:1 to 400:1).

FIGS. 7A-B. Three-dimensional structure of wild-type (A) and triple-mutant (B) AAT come from Lomas et al., indicating sites of proposed antioxidant mutations (Met351 and Met358, P8 and P1 of the active site, respectively), conservative mutation (Phe51) buried deep in the hydrophobic core of the molecule, and Cystine PEGylation (Cys232, exposed on the surface but not obstructing the active site). Modeling software COOT www2.mrc-lmb.cam.ac.uk/personal/pemsley/coot/ and PDB coordinates 1QLP were used to formulate this protein model based on solved crystal structure. The whole structure was displayed as a cartoon. The wild-type and mutant-type residues were shown in a stick form. The pictures were generated using the PYMOL software (www.pymol.org).

### DETAILED DESCRIPTION OF EMBODIMENTS

This invention provides a method for the expression and purification of the AAT mutant. Although this invention provides an expression host of E. coli., it doesn't limit the expression host range. In some embodiments, any host capable of achieving a high yield of the recombinant protein may be used to express the mutant protein. These hosts include mammalian and cellular expression hosts, plant and plant cell expression hosts, insect expression hosts, fungal expression hosts, and bacterial expression hosts. On the other hand, any carrier that can express a protein in these kinds of hosts can be used for the expression.

In some embodiments, E. coli may be used as a recombinant protein expression host. To be specific, the expression host is BL21(DE3).

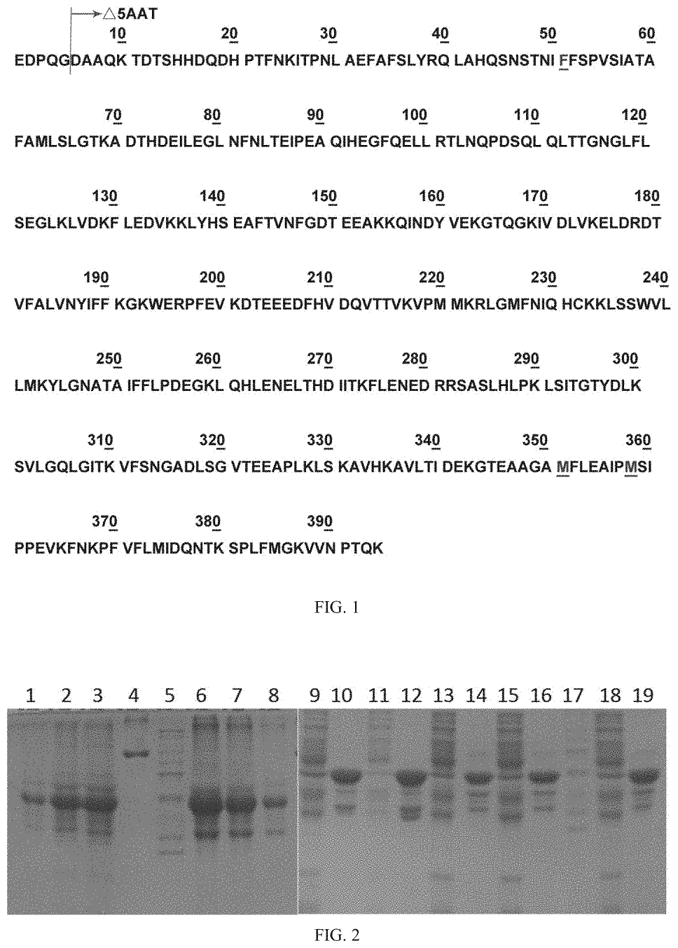

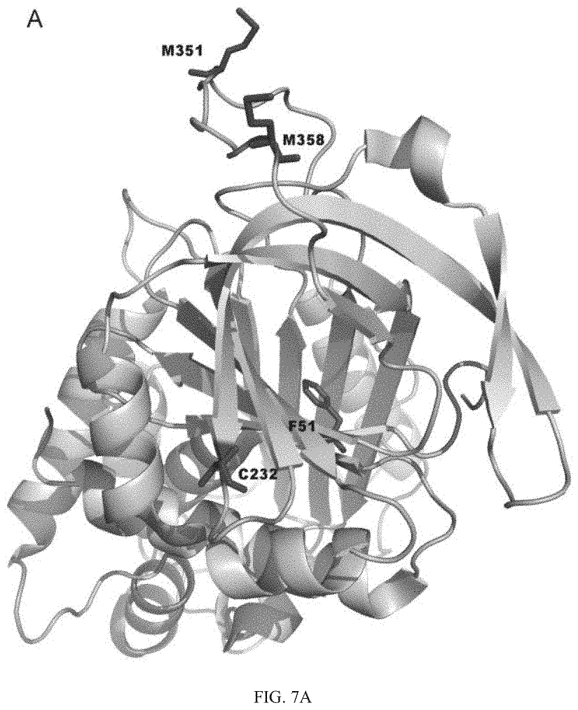

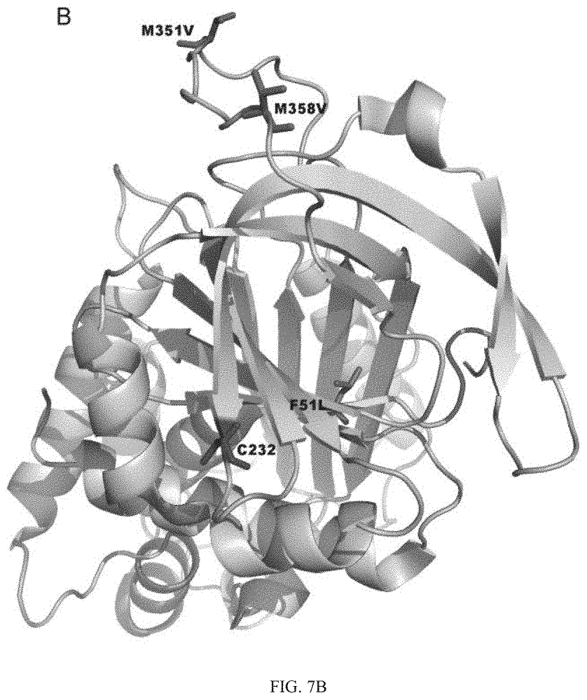

In some embodiments, pET-11 (Novagen) can be used as an expression vector in E. coli. First of all, we constructed a full-length form of AAT (FIG. 1). However, the yield was unexpectedly low. We then made N-terminal deletions and expressed the Δ5AAT (deleting the first five amino acids of the mature AAT) and Δ10AAT (deleting the first 10 amino acids of the mature AAT). Both expressed well. For practical purpose, we chose the Δ5AAT, which is better in expression and refolding efficiency than the Δ10AAT. Next, we designed multiple mutants based on the published crystal structure of the AAT protein (FIG. 7) for developing better therapeutic agents with better thermostability and anti-oxidation property. We selected three mutants for expression according to the previous screening work. The first one is the thermostable mutant F51L, the second one is the double mutant M351V/M358V designed to reduce oxidation and inactivation, and the third one is our unique triple mutant with both thermostability and antioxidation (F51L/M351V/M358V). All three muteins were constructed using standard PCR mutagenesis techniques and verified by sequencing. FIG. 1 shows the starting position of the Δ5AAT protein sequence and the specific amino acid substitution site.

In some embodiments, we screened many conditions to test E. coli expression. Finally, we found that the wild type and the selected mutants expressed well in an E. coli expression host, BL21(DE3) (FIG. 2). Our results also showed that all expression constructs could be expressed in high yields, most of which are in insoluble inclusion bodies when given appropriate growth media and proper conditions. Methods for E. coli expression and inclusion body purification had been published (X. Lin, Umetsu, T., The high pH and pH-shift refolding technology, Current Pharmaceutical Technology 11 (2010), no. 3, 293-299.). In some embodiments, the purified inclusion bodies are dissolved in a high concentration of solubilization buffer containing high concentration of chaotrophic reagents, such as a high concentration of urea buffer, for example, an 8M urea solubilization buffer and reducing agents. In other embodiments, the purified inclusion bodies are dissolved in a high concentration of guanidine hydrochloride buffer, for example, about 6M guanidine hydrochloride solubilization buffer.

In some embodiments, AAT inclusion bodies dissolved in urea or guanidine hydrochloride buffer can be further purified. For example, by column chromatography. The purification technique of inclusion bodies is well known to those skilled in the art.

The purified inclusion bodies in the solubilization buffer can be refolded in a variety of refolding buffers with different pH and chemical ingredients. In some embodiments, wild-type and mutant AAT are renatured in different refolding buffers for optimal refolding. The wild-type and mutant AAT are renatured in the same refolding buffer in certain embodiments.

In some embodiments, the refolding buffer contains Tris. In certain embodiments, the refolding buffer comprises glycerol, sucrose, or any combination thereof. For example, the refolding buffer may contain about 5% to 30% glycerol (v/v, the same below), about 5% to 40% sucrose, or about 10% glycerol and about 10% sucrose. In some embodiments, the refolding buffer can contain PEG, of which the molecular weight can be approximately 200 to 20,000 Daltons, for example, the molecular weight of PEG can be about 200 Daltons or can be about 600 Daltons. In some embodiments, the refolding buffer may include detergents, such as Tween-20, Tween-80, sodium deoxycholate, sodium cholate, and trimethylamine oxide (TMSO).

In certain embodiments, the refolding method includes rapidly diluting the AAT polypeptide solution dissolved in a solubilization buffer with a refolding buffer, i.e., about 20-fold dilution. In some embodiments, the refolding method involves dialysis of the AAT polypeptide solution dissolved in a solubilization buffer using a refolding buffer. For example, dialysis with a refolding buffer is about 20 times the volume of solubilization buffer.

In some embodiments, the refolding buffer is at a high pH, for example, at pH˜9 or pH˜10. In some embodiments, the refolding buffer is initially at a high pH and then adjusted to a neutral pH after start of the refolding, for example, from pH 10 to pH˜8 or pH˜7. In certain embodiments, this approach further involves the use of a solubilization buffer to mediate the A280 to a scale of 2.0 to 10.0 (e.g., approximately 2.0 to approximately 5.0) of the dissolved AAT polypeptide solutions before diluting the dissolved AAT polypeptide with a refolding buffer.

In an exemplification embodiment, a method for producing a refolded AAT polypeptide includes: a) dissolving a denatured AAT polypeptide with a solubilization buffer, which contains 8 M urea, 0.1 M Tris, 1 mM glycine, 1 mM EDTA, 100 mM (β-mercaptoethanol, pH 10, results in a solution of dissolved AAT polypeptide; b) adjusting A280 of dissolved AAT wild-type or mutant proprotein solution to about 2.0. This solubilization buffer contains approximately 8 M urea, 0.1 M Tris, 1 mM glycine, 1 mM EDTA,10 mM 3-mercaptoethanol, 10 mM dithiothreitol (DTT), 1 mM reduced form Glutathione (GSH), pH 10; c) quickly diluting the dissolved AAT polypeptide by adding it to the refolding buffer, which is about 20 times the volume of the solubilization buffer. This refolding buffer contains 20 mM Tris, pH 10, and any one of the following 1) ˜5). 1) 5% to 30% glycerol, 2) 5% to 40% sucrose, 3) 20% glyceriol and 20% sucrose, 4) 10% glycerol and 10% Sucrose, and 5) 5% to 10% polyethylene glycol (PEG). d) Adjust the pH of the diluted dissolved AAT polypeptide to about 7.6, thereby producing a refoled AAT polypeptide. In some variations, the refolding buffer further includes 0.005% to 0.02% Tween-20 (Tween 20).

In another exemplification embodiment, a method for refolding reconstructed wild-type and mutant AAT polypeptides includes: a) Dissolving denatured AAT polypeptide with a solubilization buffer, which contains 8 M urea, 0.1 M Tris, 1 mM glycine, 1 mM EDTA, 10 mM β-mercaptoethanol, 10 mM dithiothreitol (DTT), 1 mM reduced glutathione (GSH), pH 9, resulting in a solution of dissolved AAT polypeptide; b) rapidly diluting the above-dissolved AAT polypeptide by adding it to refolding buffer, which is approximately 20 times the volume of the solubilization buffer and contains about 20 mM Tris and 10% glycerol, pH 9; c) Slowly adjusting the pH of the diluted dissolved AAT polypeptide to about 7.6, thereby producing a refolded AAT polypeptide.

In another exemplification embodiment, a method for refolding reconstructed wild-type and mutant AAT polypeptides includes: a) dissolving denatured AAT polypeptide with a solubilization buffer, which contains about 8 M urea, 0.1 M Tris, 1 mM glycine, 1 mM EDTA, 10 mM β-mercaptoethanol, 10 mM dithiothreitol (DTT), 1 mM reduced glutathione (GSH), pH 8, resulting in a solution of dissolved AAT polypeptide; b) quickly diluting the above-dissolved AAT polypeptide by adding the above-dissolved AAT polypeptide to refolding buffer, which is approximately 20 times the volume of the solubilization buffer and contains about 20 mM Tris and 10% glycerol, pH 8; c) slowly adjusting the pH of the diluted dissolved AAT polypeptide to about 7.6, thereby producing a refolded AAT polypeptide.

In another exemplification embodiment, a method for refolding reconstructed wild-type and mutant AAT polypeptides includes: a) Dissolving denatured AAT polypeptide with a solubilization buffer, which contains about 8 M urea, 0.1 M Tris, 1 mM glycine, 1 mM EDTA, 10 mM β-mercaptoethanol, 10 mM dithiothreitol (DTT), 1 mM reduced glutathione (GSH), pH 7.6, results in a solution of dissolved AAT polypeptide;

b) Rapidly dilute the above-dissolved AAT polypeptide by adding it to refolding buffer, which is 20 times the volume of the solubilization buffer and contains about 20 mM Tris and about 10% glycerol, pH 8;

c) Slowly adjusting the pH of the diluted dissolved AAT polypeptide to about 7.6, thereby producing a renatured AAT polypeptide.

In certain embodiments, this method further includes a protocol for concentrating renatured wild-type and mutant AAT polypeptides. For example, an ultrafiltration method can be used to concentrate the renatured AAT polypeptide 10-200 times.

The invention also provides a method for purifying correctly renatured wild-type and mutant AAT polypeptides from incorrectly folded or non-renatured AAT. This method includes:

a) Incorrect renatured or non-renatured AAT peptides are bound to hydrophobic chromatography resins in the presence of salt;

b) Collect the correct renatured AAT polypeptides that are not binding to the resin.

In some embodiments, the salt is ammonium sulfate [(NH4) 2SO4], or sodium chloride (NaCl), or potassium chloride (KCl). In some embodiments, the concentration of ammonium sulfate ranges from about 0.25 M to about 1.2 M. In some embodiments, the concentration of sodium chloride ranges from about 1.0 M to about 3.5 M. In some embodiments, the concentration of potassium chloride ranges from about 1.0 M to about 3.5 M. In some embodiments, the appropriately renatured AAT polypeptides are derived from bacterial inclusion bodies.

In an illustrative implementation, the method for producing renatured recombinant wild-type and mutant AAT polypeptides includes: a) Dissolving the denatured AAT polypeptide with a solubilization buffer, which contains about 8 M urea, 0.1 M Tris, 1 mM glycine, 1 mM EDTA, 100 mM β-mercaptoethanol, pH 9.0, resulting in a dissolved AAT polypeptide solution. b) Adjust A280 of the dissolved AAT protein solution with a solubilization buffer to about 2.0. This solubilization buffer contains approximately 8 M urea, 0.1 M Tris, 1 mM glycine, 1 mM EDTA, 10 mM (3-mercaptoethanol, 10 mM dithiothreitol (DTT), 1 mM reduced form Glutathione (GSH), pH 9.0. c) Quickly dilute the above-dissolved AAT polypeptide by adding it refolding buffer, which is about 20 times the volume of the solubilization buffer. The refolding buffer contains about 20 mM Tris, pH 9.0, and any one of the following reagents 1) 5): 1) about 10% to about 30% glycerol, 2) about 10% to about 50% sucrose, 3) about 20% glycerol and about 20% sucrose, 4) about 10% glycerol and about 10% sucrose, and 5) about 5% to about 10% polyethylene glycol (PEG). d) Incubate the diluted dissolved AAT polypeptide solution at 20° C. for at least 16 hours. e) The diluted dissolved AAT polypeptide solution is further incubated at 4° C. for about 24 to about 72 hours. f) The diluted dissolved AAT polypeptide solution is concentrated by ultrafiltration. And g) The diluted dissolved AAT polypeptide solution was exchanged by a size exclusion chromatography (SEC) (Superdex 75, Superdex 200, or Sephacryl S-300, GE Healthcare) into a renatures AAT polypeptide with a buffer containing 20 mM Tris, 0.2 M NaCl, 10% glycerol or 15% sucrose, 1 mM DTT, pH 7.6, thereby producing a renatured AAT polypeptide. In certain embodiments, the refolding buffer and the buffer in step g) further comprise about 0.005% Tween-20 (Tween 20).

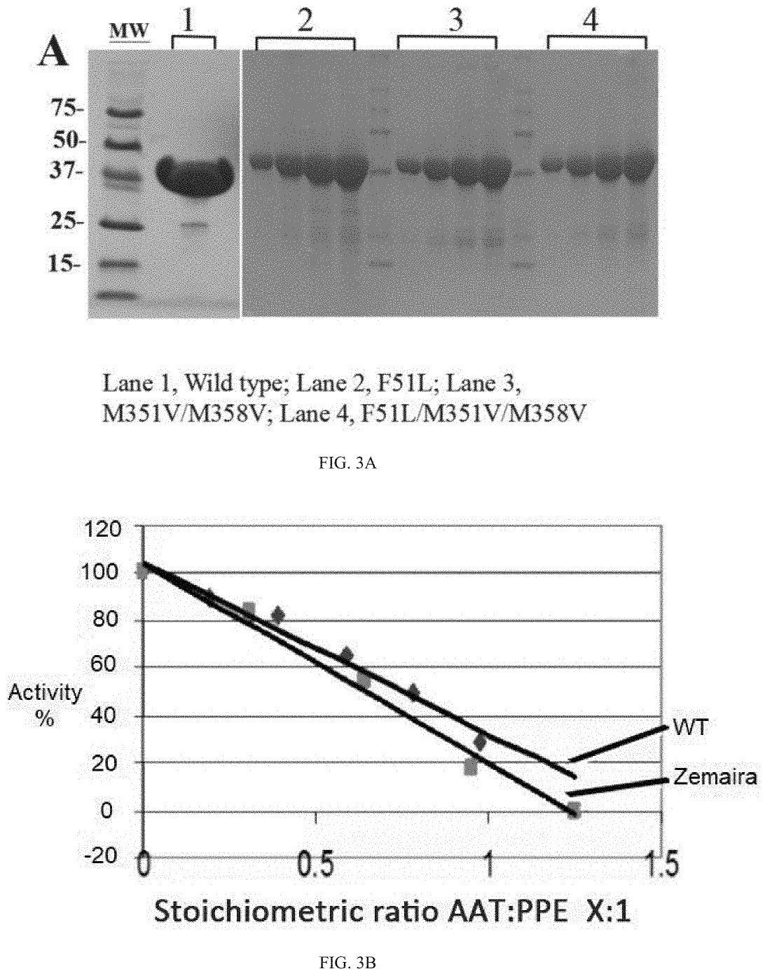

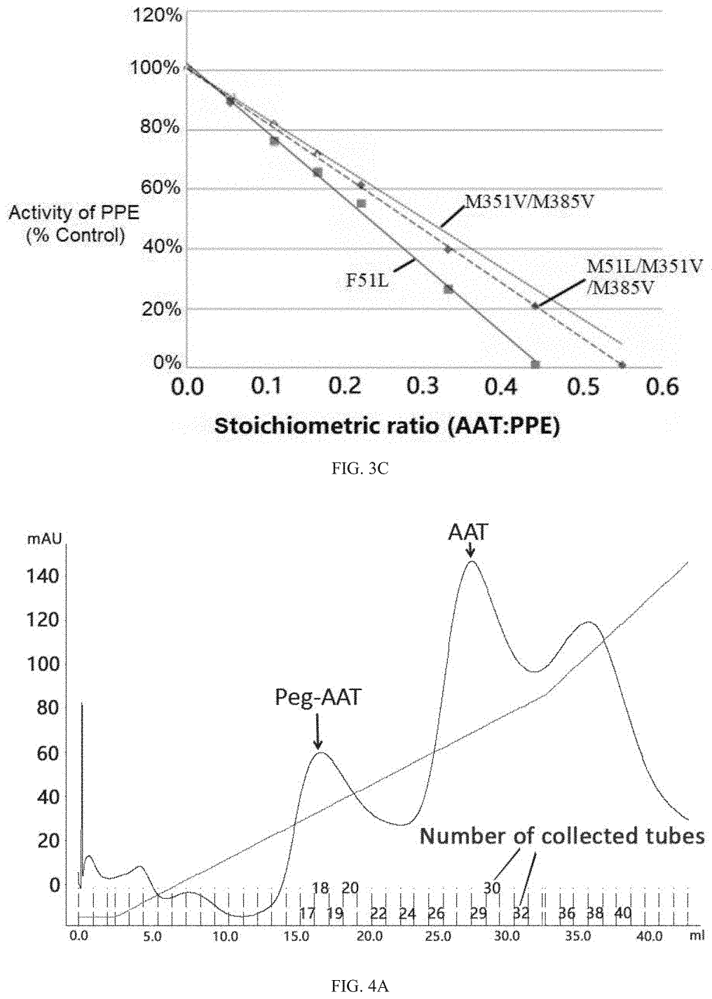

In another illustrative implementation scheme, the purification steps for the renatured wild type and mutant AAT includes:1) Concentrate the renatured solution by ultrafiltration. And then separate the refolded monomeric protein from non-refolded or partially refolded AAT through a SEC column. 2) Refolded proteins were further purified by ion-exchange or hydrophobic interaction column chromatography. FIG. 3A shows the SDS-PAGE result of purified AAT samples obtained by hydrophobic interaction chromatography. It shows that most of the protein is in monomeric form, but a small amount of dimer form shows up in the absence of a reducing agent. Non-reducing SDS-PAGE has been routinely used to distinguish between folded and unfolded proteins. The purified recombinant AAT shows almost the same inhibitory activity as native human AAT (glycosylated Zemaira® from Aventis Behring LLC, available for clinical treatment). At a stoichiometric ratio of AAT: PPE of approximately 1.07:1, PPE (porcine pancreatic elastase) is completely inhibited (FIG. 3B), indicating that the purified recombinant AAT is fully active. The purified mutant protein (mutein) is shown in FIG. 3C. The activity of the mutant protein is comparable to that of the wild type.

### Chemical Modification

Chemical modification of protein drugs is a standard method to improve in vivo half-life. The AAT protein has a unique cysteine at position 232 (FIG. 7). This site (Cys232) or N-terminal site can be used for site-specific chemical modification, and it has been proved that the modification does not affect the activity of AAT.

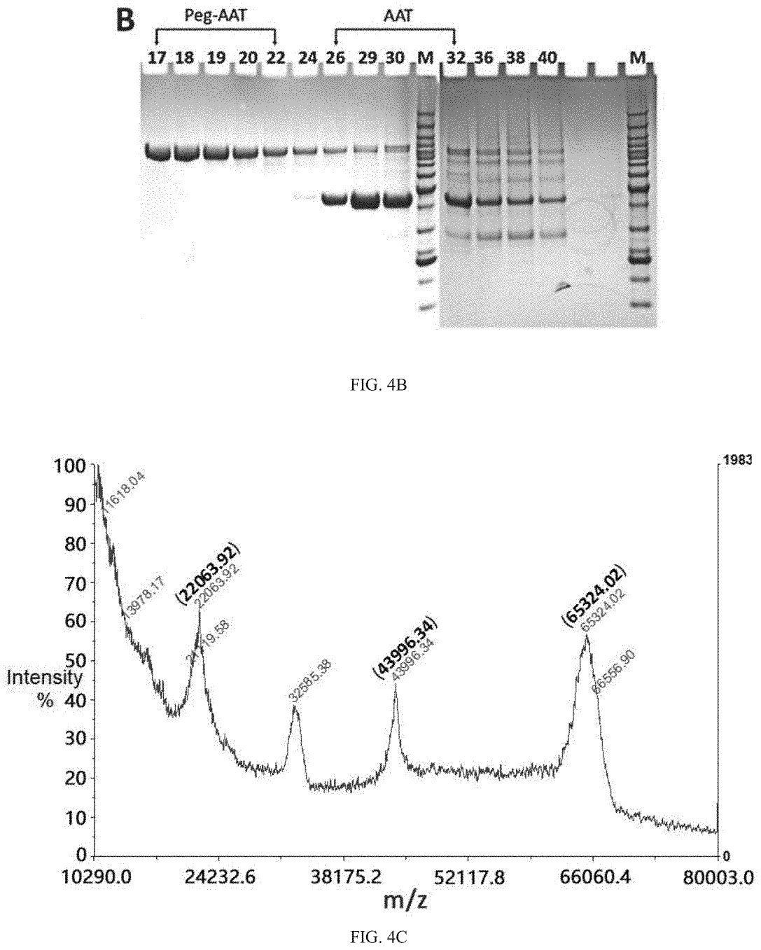

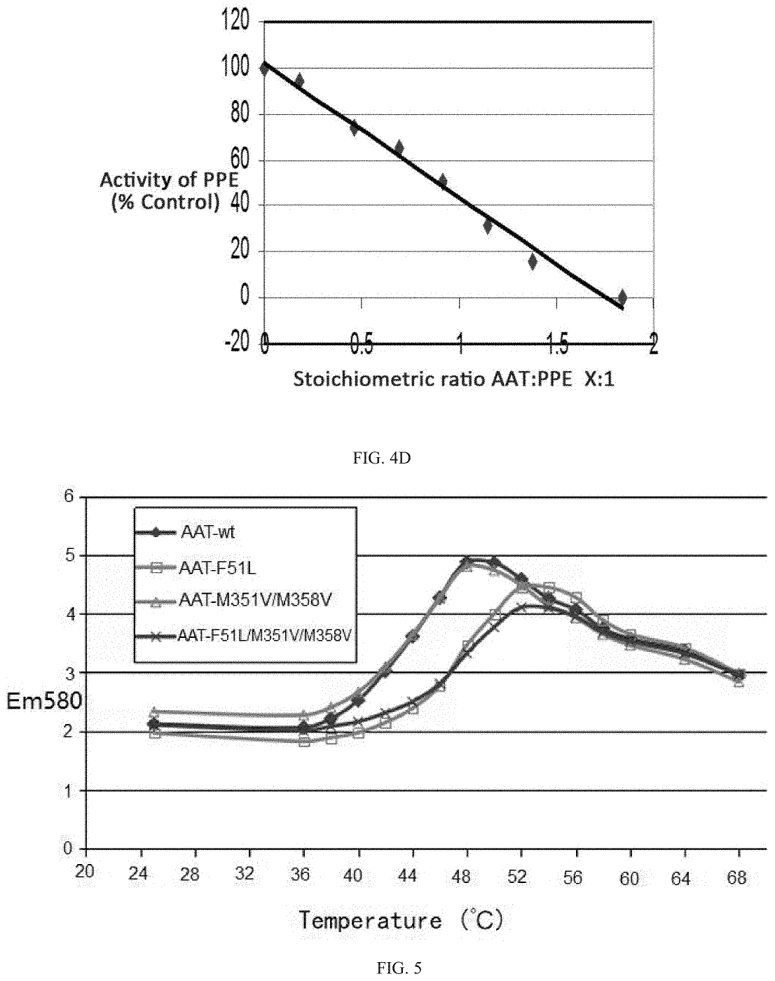

In certain embodiments, PEGylation is performed at the Cys232 site. According to the published methods, purified rAAT can be pegylated at the unique Cys232 site. The efficiency of PEGylation is in the range of 50-65% in multiple experiments. The molecular model indicates that this unique cysteine is partially exposed to aqueous solvents and is not near the AAT domain that interacts with elastase (see FIG. 7). After PEGylation, unreacted maleimide-PEG (˜20K Da, Nektar Therapeutics) and non-polyethylene AAT were separated from the pegylated AAT by anion exchange chromatography (Q-HiTrap, GE Healthcare). FIG. 4 shows SDS-PAGE and MALDI-TOF mass spectrometry results of pegylated rAAT peptides and its normal function in blocking PPE. The results show that pegylated AAT can be easily separated from non-pegylated AAT and free unreacted mPEG20 by salt gradient elution. Moreover, the success of the PEGylation reaction has been confirmed by SDS-PAGE and MALDI-TOF mass spectrometry. For example (FIG. 4C), the molecular weight of the AAT polypeptide (non-pegylated) is 43996.34 Daltons, while the molecular weight of Mal-PEG20 is 22063.92 Daltons. The molecular weight of the successfully pegylated rAAT is 65324.02 Daltons, which closely matches the predicted molecular weight. The results indicated that rAAT has been successfully pegylated. In addition, “dissociated” rAAT and Mal-PEG20 masses may be produced during the ionization decomposition of the mass spectrometry.

In certain embodiments, the fatty acid modification at the Cys232 site can be performed, one of which is palmitoyl modification. Palmitic acid is a hexadecane fatty acid. In blood, palmitic acid has a strong binding ability with serum albumin. Using this character, palmitic acid-modified protein drugs can be made to bind serum albumin in the blood, greatly prolonging the in vivo half-life. Palmitic acid-modified protein or peptide drugs have been successfully used in clinics and performed very well. Among them, Novo Nordisk's Liraglutide is a successful example, which is an approved diabetes and obesity drug. This invention provides wild-type and mutant AAT proteins modified with palmitic acid at the Cys232 site. The following formula is a modification that uses glutamic acid as a linker or “bridge” to connect palmitic acid to wild-type or mutant AAT-Cys232.

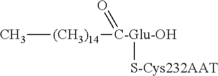

Other chemical linking methods can also be used. For example, under specific chemical reaction conditions, N-terminal chemical modification (Christopher D. Spicer & Benjamin G. Davis Nature Communications 5, Article number: 4740 (2014). “Selective chemical protein modification”) is well known to those skilled in the art.

### Thermostability

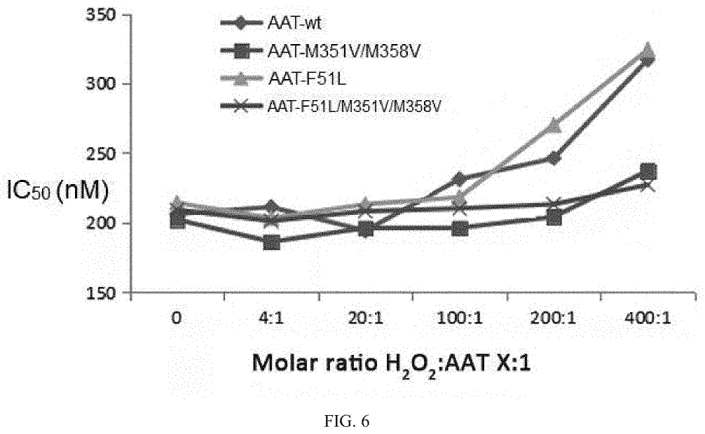

For improving of the thermostability and oxidation resistance of recombinant AAT, three mutants were constructed, of which the first is a thermostable F51L single mutant, the second is an antioxidant M351V/M358V double mutant, and the third is a thermostable and antioxidant F51L/M351V/M358V triple mutant. To compare the thermostability of wild-type and mutant proteins (muteins), we used a fluorescence-based thermal denaturation assay, as shown in FIG. 5. In this case, the SYPRO Orange dye binds to hydrophobic surface of a protein. When the test protein denatured as temperature increases, its hydrophobic surface is exposed and bound to the dye, resulting in increased fluorescence. Further increasing the temperature separated the dye from protein and produced a denaturation peak. FIG. 5 showed that the wild-type and M351V/M358V mutant proteins undergone thermal denaturation at around 48° C., while the denaturation temperature of F51L, and F51L/M351V/M358V mutant proteins all rose to around 54° C. The results showed that the single and triple mutants containing F51L have greatly improved thermostability.

### Oxidation resistant

In addition to constructing the antioxidant mutant M351V/M358V, a combination mutant of F51L/M351V/M358V was also constructed. It was predicted that both the oxidation resistance and thermostability would be improved. As mentioned above (FIG. 5), the three mutants were indeed more thermostable. In the anti-oxidation experiments, the results showed that the two M351V/M358V-containing mutant proteins were more resistant to oxidation. As shown in FIG. 6, when the molecular ratio of H2O2 to AAT increased from 4:1 to 400:1, natural AAT and F51L began to lose the activity of inhibiting PPE in vitro, but the oxidation resistance of mutant proteins containing M351V/M358V could reach 400:1 ratio. According to reports in the literature, as with heat-stable mutants, the in vitro antioxidant properties of muteins can be translated into enhanced in vivo stability, and therefore have improved “pharmaceutical properties”.

### Pharmaceutical Compositions and Kits

This invention also provides the AAT polypeptide composition (including pharmaceutical composition), compromising biological activity. The composition may also contain pharmaceutical excipients. The AAT polypeptide may be in the form of a lyophilized preparation or a liquid preparation. Pharmaceutical excipients are non-toxic to the patients at the dosage and concentration used and may contain buffers such as phosphates and citrates, and salts such as sodium chloride, and sugars such as sucrose, and/or polyethylene glycol (PEG). See Remington: The Science and Practice of Pharmacy 20th Ed. (2000) Lippincott Williams and Wilkins, Ed. KE Hoover. AAT polypeptide can be prepared by different administration routes, such as intravenous (IV) liquid or lyophilized preparations, and dry powder preparation or gasification preparation for deep lung administration. These reagents are evident to those skilled in the art. The reference list includes Drug Delivery to the Lung, Bisgaard H., O'Callaghan C, and Smaldone GC editors, New York; Marcel Dekker, 2002.

The AAT polypeptide in this invention can be produced by any method described herein. In some embodiments, AAT polypeptides are produced from the inclusion bodies of bacteria (e.g., E. coli). In some embodiments, the AAT polypeptide is non-glycosylated. In some embodiments, the AAT polypeptide has a purity of at least about 80%, 85%, 90%, 95%, 96%, 97%, 98%, or 99%. In some embodiments, the specific activity of the AAT polypeptide (e.g., determined by porcine pancreatic elastase inhibition) is not less than about 0.3, 0.35, 0.4, 0.45, 0.5, 0.55, 0.6, 0.65, 0.7, 0.75 0.8, 0.85, 0.9 or 0.95 mg of active AAT polypeptide per mg of total protein. In some instances, the AAT polypeptide is a wild-type AAT protein. In some examples, the AAT polypeptide is a mutant AAT protein. In some examples, the AAT polypeptide is a high-stability, oxidation-resistant triple mutant AAT described in the present invention. In certain instances, the AAT polypeptide is a chemically modified AAT protein described above.

This invention for therapeutic application comprising the AAT polypeptide provides a kit, which comprises one or more containers containing AAT polypeptide. These containers can be a small potion bottle, bottle, jar, or flexible packaging. For example, the AAT polypeptide can be packaged in a small disposable vial containing 500 mg or 1,000 mg of active AAT polypeptide. The vial can have a sterile access port (for example, a stopper that can be pierced by a hypodermic needle). Other expected packages are combined with special devices, such as inhalers, nasal drug delivery devices (e.g. nebulizers), or input devices such as micropumps. At least one active agent is an AAT polypeptide. The kit may further include the active ingredient of the second medicine. The packaging container may also contain an application manual according to the method described in this invention. Generally, these instructions include instructions for the application of AAT polypeptide in the treatment of diseases according to the methods described in this invention. The instructions may further include descriptions for using AAT polypeptides to treat diseases, for instance, diseases associated with AAT deficiency. The instructions generally include the dosages, duration of use, and the route of application for the treatment of the disease. The instructions provided in the kit of this invention are generally written on a label or instructions (for example, on the paper contained in the kit), and machine-readable instructions (for example, instructions on a magnetic disk or a compact disc) are also acceptable. The kit may also include a device for pulmonary administration of dry powder or nebulizer.

The following illustrations provide examples, but not to limit, the invention.

Example 1. Plasmid construction and expression. The DNA fragment encoding Δ5-AAT polypeptide (FIG. 1) was obtained by PCR amplification. Δ5-AAT polypeptide lacks the first 1-5 amino acid sequence shown in FIG. 1 and is artificially added a methionine at the starting position to promote expression in E. coli. The codon sequence in the DNA fragment encoding the Δ5-AAT polypeptide had been optimized for optimal expression in E. coli. For protein expression, the above PCR product was cloned into a pET11a plasmid. After PCR, ligation, and transformation into the BL21 (DE3) strain, single colonies were selected for expression. Furthermore, DNA sequencing was performed on the selected vector to ensure that the correct DNA sequence is obtained. The resulting vector is pET11-Δ5-AAT.

Example 2. Expression of wild-type and F51L mutant proteins. First of all, the E. coli expression clones were expanded and then inoculated into 1 L LB medium containing 10 g tryptone, 5 g yeast extract, 10 g NaCl, and 50 mg ampicillin. When OD600 of reached to about 0.6, IPTG was added to a final concentration of 0.5 mM. The bacteria were continued to grow at 37° C. for another 3 hours.

Example 3. Expression of M351V/M358V and F5IL/M351V/M358V muteins. First, the E. coli expression clone was amplified in LB medium, then inoculated into 1 L medium containing 12 g tryptone, 24 g yeast extract, 4 mL glycerol, 17 mM KH2PO4, 72mM KH2PO4, and 50 mg ampicillin. When OD600 reached to about 0.6, IPTG was added to a final concentration of 0.5 mM. The culture was then grown at 37° C. for another 3 hours.

Example 4. Purification of inclusion bodies. The cells were collected by centrifugation and then suspended in 20 mL TN buffer (150 mM NaCl, 50 mM Tris, pH 8.0) containing 1% Triton-X-100. 10 mg of lysozyme was added thereto, and the cells were suspended and frozen at −20 ° C. overnight. The lysate was then dissolved, and 20 μl of 1 M magnesium sulfate and 100 μL of 0.01 mg/mL DNAase were added. The cells were agitated and incubated at room temperature until the released DNA was completely dissolved. After that, the lysate was diluted with 250 mL of TN containing 1% Triton-X-100, and mixed by stirring for 2-4 hours. The inclusion bodies were collected by centrifugation and purified by washing 5 times with TN buffer (100 mM Tris, 250 mM NaCl, pH 8.0) containing 1% Triton X-100. The purified inclusion bodies were dissolved in 8M urea buffer solution (8 M urea, 0.1 M Tris, 1 mM glycine, 1 mM EDTA, 100 mM β-mercaptoethanol, pH 10), and slowly stirred at 4° C. for about 16 hours. The lysate was then centrifuged to remove insoluble debris. The purified inclusion bodies were adjusted to a final A280=2.0 by using the same 8 M urea buffer as the diluent.

Example 5. Refolding. The solubilized inclusion bodies were quickly diluted to 20 volumes of buffer containing: 20 mM Tris, 10% glycerol, pH 9, and the final OD280 after dilution was 0.1. After dilution, the pH of the solution was gradually adjusted to 7.6 within 2 to 4 days with 1 M HCl. Other refolding methods that have been tested include the usage of a high concentration of glycerol (20%) in the buffer, or replacement of glycerol with 20% sucrose, or simultaneous use of 10% sucrose and 10% glycerol. In some embodiments, Tween-20 (0.005% -0.01%) is also included in the refolding buffer. All these conditions produce the correct refolded (active) AAT polypeptide.

The expression of wild-type and mutant-type AAT polypeptide inclusion bodies can also be successfully renatured by a fixed pH method. The washed inclusion bodies were dissolved in a solubilization buffer containing a high concentration of urea (8 M urea, 0.1 M Tris, 1 mM glycine, 1 mM EDTA, 100 mM β-mercaptoethanol, pH 10.5), dissolved in high OD280 (20-40) and agitated slowly at 4° C. for 12 hours. The dissolved sample was clarified by ultracentrifugation (30 minutes×66,000 g) to remove undissolved impurities. Then with buffer containing 8M urea, 0.1 M Tris, 1 mM glycerol, 1 mM EDTA, 10 mM β-mercaptoethanol, 10 mM dithiothreitol (DTT), 1 mM reduced glutathione (GSH)), pH 10.5, the OD280 of the dissolved inclusion bodies was adjusted to 2.0. The solubilized inclusion bodies were quickly diluted to 20 volumes of buffer containing 20 mM Tris, 10% glycerol, pH 8.5, and the final OD280 after dilution was 0.1. The diluted solution was stored at 20° C. for 16 hours and then subjected to ultrafiltration concentration and buffer exchange.

Example 6. Purification. The refolded AAT solution was concentrated to A280>20.0 using a tangential flow ultrafiltration system and load into Superdex 200 column pre-equilibrated with a buffer containing 20 mM Tris, 0.15M NaCl, 0.4M urea, 1 mM DTT, 10% glycerol, pH 7.6. The active peak fraction was collected and dialyzed against a buffer containing 20 mM Tris, 5% glycerol, 3M NaCl, 0.001% Tween, 20, 1 mM DTT, pH 7.6. The dialyzed sample were then loaded onto a phenyl agarose gel (hydrophobic) column equilibrated with the dialysis buffer. The effluent containing the purified product of interest was collected, concentrated, and dialyzed with a buffer containing 20 mM Tris, 5% glycerol, 0.001% Tween 20, 1 mM DTT, pH 7.6. The protein concentration is determined with the molar extinction coefficient in 6 M guanidine hydrochloride, 20 mM sodium phosphate, pH 6.5. For this particular protein, the extinction coefficient ∈280=19060 M-1 cm-1.

Example 7. PEGylation. DTT was removed from the highly purified AAT by a PD-10 (BioRad) column pre-balanced with 50mM sodium phosphate (pH7.5) and 200mM NaCl, and the pH was adjusted to 7.5 according to product requirements. Since the reducing agent DTT interferes with the PEGylation reaction, twice buffer exchange processes are usually performed to ensure that no trace of DTT is present. AAT after exchange buffer was quantified by molar extinction. Solid PEG-ma120 (polyethylene glycol maleimide 20, Nektar, Huntsville, Ala.) stored in argon at −20° C. was then added to the AAT solution at a molar ratio of 5:1 to 10:1, and incubated at 37° C. for 30 minutes. The reaction was terminated by adding 20 mM DTT and incubated at 37° C. for 5 minutes. The PEGylated AAT (Peg-AAT) was then dialyzed into 20 mM Tris, 50 mM NaCl, 1 mM DTT pH 8 to remove excess salt, then loaded onto a 5 mL HiTrap Q XL column for gradient elution with 0-1000 mM NaCl.

Example 8. Enzyme activity determination. The AAT biological activity of refolding wild-type rAAT and mutant AAT was measured in vitro by chromogenic substrate reaction against HLE or PPE inhibitory activity. We tested the inhibitory activity of rAAT and compared it with commercially available human plasma AAT (San Diego, Calif. catalog #17825) produced by Calbiochem or commercially available full-length glycosylated AAT produced and sold by Aventis Behring LLC. PPE isolated from live pig pancreas was purchased from Sigma-Aldrich (St. Louis, Mo., Catalog # E7885); HLE isolated from human sputum was purchased from Molecular Innovations (Southfield, Mich. Cat # HNE). The concentration of AAT ranges from 0.3 nM to 14 nM was incubated at 37° C. for 15 minutes mixed with a fixed concentration of 1.4 nM PPE or HLE. And then, an aliquot of the incubation was incubated with 1mM elastase substrate N-succinyl-ala (PPE chromogenic substrate, Sigma) or N-methoxysuccinyl-α-alanyl-α-alanyl-p-nitroaniline (HLE chromogenic substrate, Sigma). The kinetics of the substrate hydrolysis was measured by a Molecular Devices spectrophotometer (Spectramax Plus) at 21° C. and 405 nm, to determine the initial rate of each reaction and calculate the percentage of activity relative to the control group (without AAT or AAT polypeptide). The stoichiometric molar ratio of the percentage of elastase activity relative to the concentration of AAT polypeptide/elastase used in the corresponding reaction is plotted. The accurate concentration of each form of AAT polypeptide, PPE and HLE stocks used in the experiment was determined in advance by a known extinction coefficient, which was obtained from the computer of the ExPASY proteomics server of the Swiss Bioinformatics Institute The software program ProtParam (http://www.expasy.ch).

The detailed experimental process of FIGS. 3A-C is listed below. The PPE with a fixed concentration (80 μg/mL) was added to Eppendorf tubes with different concentrations of AAT, then incubated at 37° C. in the reaction system of 50 mM Tris pH 8.8, 38 mM NaCl, and 0.01% Tween 20 for 15 minutes. Pipette 10 μL aliquots in quadruplicate into a microtiter plate, then use a multichannel pipette to pipette 100 μL aliquots of 1 mM chromogenic substrate (P-α-alanine-pro-val-pNA) into the well of the microplate in the same buffer solution. The kinetics of elastase cleavage of the substrate was monitored at 405 nm at 21° C., to made a comparison of reaction rate with a control group (elastase only) results in a series of percentages, and plot the relative value (%) of the protease (y-axis) against the stoichiometric ratio of AAT:PPE (x-axis). The concentration of PPE used in the assay was measured by measuring the extinction coefficient of pure PPE in 6M guanidine, 50 mM NaPi, pH6.5, according to the ProtParam algorithm of the Swiss Bioinformatics Institute (www.expasy.ch). The concentration of AAT is determined by the following method. First, the concentration of trypsin (Sigma) active site was titrated accurately with the “nearly irreversible” fluorescent substrate MUGB (Fluka) from Novagen (www.novagen.com). And then, the chromogenic substrate BAPNA (n-benzoyl 1-arg-4 nitroaniline hydrochloride, Sigma) was used to determine the concentration of any AAT solution at the functional site of blocking trypsin in the stoichiometric analysis at 21° C. and 405 nm. The active concentration of either form of AAT has been determined to be almost the same as that determined by using a ProtParam computer algorithm from the Swiss bioinformatics research institute website (www.expasy.ch) to measure the functional concentration structurally using the extinction coefficient, indicating that the activity of the purified recombinant AAT is nearly 100%.

Example 9. Thermostability. The thermostability measurement was performed using a 96-well culture plate. The reaction volume was 110 μL, and the buffer contained 1×PBS buffer, 10% (v/v) glycerol, 10% DMSO, 5 mM DTT, 50×SYPRO Orange and 15 μM each of purified AAT or its mutant. The reaction culture plate was incubated at 25° C. for 30 min and then heated to 70° C. at 0.5° C. intervals. Ex 490 mM at each temperature and 200 mS fluorescence at Em 580 mM were measured. The figure was made by plotting the fluorescent count against temperature.

Example 10. Oxidation resistance. For testing of the oxidation resistance of AAT and its mutants, 50 μM of each purified AAT or mutants were incubated in PBS buffer containing 0 mM, 2 mM, 10 mM, 50 mM, 100 mM, and 200 mM H2O2 for 15 minutes at 25° C. The same amount of DTT was then added to reduce the excess H2O2. The oxidation resistance of the treated AAT and mutants was determined by measuring the inhibitory activity against PPE.

We claim:

1. A recombinant mutant al-antitrypsin (AAT) polypeptide, wherein the polypeptide is a triple mutant F51L/M351V/M358V AAT polypeptide and/or a chemically modified form of α1-antitrypsin (AAT) polypeptide, having a chemical modification on cysteine at position 232 (Cys232) of wild-type or mutant AAT polypeptide, wherein the chemical modification on Cys232 comprises PEGylation or a fatty acid modification; wherein the fatty acid modification comprises Palmitoylation.

2. The recombinant mutant AAT polypeptide of claim 1, wherein the recombinant mutant AAT polypeptide comprises an amino acid sequence of SEQ ID NO:1, or an amino acid sequence with a deletion of one or more N-terminal amino acid residues of SEQ ID NO:1.

3. The recombinant mutant AAT polypeptide of claim 3, wherein the recombinant mutant AAT polypeptide comprises an amino acid sequence with a deletion of N-terminal amino acid residues at positions 1-5 of SEQ ID NO:1, or with a deletion of N-terminal amino acid residues at positions 1-10 of SEQ ID NO:1.

4. The recombinant mutant AAT polypeptide of claim 1, wherein the chemically modified form comprises a chemical modification at a specific site of the wild-type or F51L/M351V/M358V triple mutant.

5. The recombinant mutant AAT polypeptide of claim 4, wherein the chemical modification is performed at Cys232 or an N-terminal site.

6. The recombinant mutant AAT polypeptide of claim 4, wherein the chemical modification comprises PEGylation or a fatty acid modification; wherein the fatty acid modification comprises Palmitoylation.

7. A method for preparing the recombinant mutant AAT polypeptide of claim 2, comprising the following steps:
1) constructing a gene encoding the recombinant mutant AAT polypeptide into an expression vector to express a recombinant protein through an expression host;
2) collecting and purifying inclusion bodies containing the recombinant protein;
3) dissolving the inclusion bodies with a solubilization buffer and then renaturing the recombinant protein with a refolding buffer; and
4) purifying the refolded recombinant protein.

8. The method of claim 7, wherein in step 1), the expression host is E. coli to overexpress the mutant protein; and in step 3), the solubilization buffer contains a high concentration of urea or guanidine hydrochloride, and the refolding buffer is a Tris buffer containing glycerol, sucrose and/or polyethylene glycol.

9. The method of claim 8, wherein the refolding buffer further comprises a detergent, which contains one or more of the following reagents: Tween-20, Tween -80, sodium deoxycholate, sodium cholate, and trimethylamine oxide.

10. The method of claim 7, wherein the step 3) is performed by any one of the following methods 1 to 4:
method 1 comprising: a) dissolving the inclusion bodies with a first solubilization buffer containing 6-8 M urea, 0.01-0.1 M Tris, 1 mM glycine, 1 mM EDTA, 10-100 mM β-mercaptoethanol, pH 7-10, to obtain a solubilized polypeptide solution; b) adjusting A₂₈₀ of the solubilized polypeptide solution to 1.0-4.0 with a second solubilization buffer, which contains 6-8 M urea, 0.01-0.1 M Tris, 1 mM glycerol, 1 mM EDTA, 1-10 mM β-mercaptoethanol, 1-10 mM dithiothreitol, 1 mM reduced glutathione, pH 8-10; c) rapidly diluting the resulting solution obtained from b) with a refolding buffer 10-50 times the volume of the solution, which contains 1-20 mM Tris, pH 7-10 and any one of the following reagents I) to V): I) 5% to 30% glycerol, II) 5% to 40% sucrose, III) 20% glycerol and 20% sucrose, IV) 10% glycerol and 10% sucrose, V) 5% to 10% polyethylene glycerol; and d) adjusting pH of the diluted solution to 7.0-8.5, thereby producing a refolding mutant protein;
method 2 comprising: a) dissolving the inclusion bodies with a solubilization buffer containing 6-8 M urea, 0.01-0.1 M Tris, 1 mM glycine, 1 mM EDTA, 1-10 mM 3-mercaptoethanol, 1- 10 mM dithiothreitol, 1 mM reduced glutathione, pH 8-10, to obtain a solubilized polypeptide solution; b) rapidly diluting the solubilized polypeptide solution with a refolding buffer 10-50 times the volume of the solubilized polypeptide solution, which contains 1-20 mM Tris and 5-30% glycerol, pH 8-10; and c) slowly adjusting pH of the diluted solution to 7.0-8.5, thereby producing a refolded mutant protein;
method 3 comprising: a) dissolving the inclusion bodies with a solubilization buffer containing 6-8 M urea, 0.01-0.1 M Tris, 1 mM glycine, 1 mM EDTA, 1-10 mM β-mercaptoethanol, 1-10 mM dithiothreitol, 1 mM reduced glutathione, pH 8, to obtain a solubilized polypeptide solution; b) rapidly diluting the solubilized polypeptide solution with a refolding buffer 10-50 times the volume of the solubilized polypeptide solution, which contains 1-20 mM Tris and 5-30% glycerol, pH 8; and c) slowly adjusting pH of the diluted solution to 7.6, thereby producing a refolded mutant protein;
method 4 comprising: a) dissolving the inclusion bodies with a solubilization buffer containing 6-8 M urea, 0.01-0.1 M Tris, 1 mM glycine, 1 mM EDTA, 1-10 mM 3-mercaptoethanol, 1-10 mM dithiothreitol, 1 mM reduced glutathione, pH 7.6, to obtain a solubilized polypeptide solution; b) rapidly diluting the polypeptide solution with a refolding buffer 10-50 times the volume of the polypeptide solution, which contains approximately 20 mM Tris and 10% glycerol, pH 7.6, thereby producing a refolded mutant protein.

11. The method of claim 7, wherein the step 3) comprises: a) dissolving the inclusion body with the first solubilization buffer, which contains 6-8 M urea, 0.01-0.1 M Tris, 1 mM glycine, 1 mM EDTA, and 10-100 mM β-mercaptoethanol, pH 9.0, to obtain a solubilized polypeptide solution; b) adjusting A₂₈₀ of the solubilized polypeptide solution to 1-4 with the second solubilization buffer, which contains 6-8 M urea, 0.01-0.1 M Tris, 1 mM glycine, 1 mM EDTA, 1-10 mM β-mercaptoethanol, 1-10 mM dithiothreitol, 1 mM reduced glutathione, pH 9.0; c) rapidly diluting the solution obtained in b) with a refolding buffer 10-50 times the volume of the solution obtained in b), which contains 1-20 mM Tris, pH 9.0, and any one of reagents of I) to V) listed below: I) 5% to 30% glycerol, II) 5% to 50% sucrose, III) 20% glycerol and 20% sucrose, IV) 10% glycerol and 10% sucrose, V) 5% to 10% polyethylene glycol; d) incubating the diluted solution at about 20° C. for about 16 hours; e) incubating the diluted solution at about 4° C. for about 24 to 72 hours; f) concentrating the diluted solution by ultrafiltration; and g) exchanging the concentrated solution by SEC to a buffer containing 10-20 mM Tris, 0.1-0.2 M NaCl, 5-30% glycerol or 5-40% sucrose, 1 mM DTT, pH 7.6, thereby producing a refolded mutant protein.

12. The method of claim 11, wherein the buffer in step g) further comprises about 0.005% Tween-20.

13. The method of claim 7, wherein the step 4) comprises separating the incorrectly refolded or non-refolded from correctly folded mutant protein by a hydrophobic interaction column chromatography in the presence of a high salt solution, and purifying the incorrectly folded protein binding to the column and the correctly refolded mutant protein by passing through the column.

14. The method of claim 13, wherein the high salt solution contains any one of ammonium sulfate , sodium chloride and potassium chloride, and a concentration of ammonium sulfate is 0.25 M to 1.2 M, a concentration of sodium chloride is 1.0 M to 3.5 M, and a concentration of potassium chloride is 1.0 M to 3.5 M.

15. The method of claim 7, wherein the step 4) comprises a step of ultrafiltration to concentrate the refolding solution and running an SEC column to separate the correctly folded monomer protein from the non-refolded or partially refolded protein;
and a step of using ion exchange or/and hydrophobic interaction column chromatography to purify the refolding protein.

16. The method of claim 7, wherein the purified refolded mutant protein obtained in step 4) is further chemically modified in a unique cysteine site of AAT.

17. A pharmaceutical composition comprising the recombinant wild-type or mutant AAT polypeptide of claim 1 or a chemically modified form thereof, and a pharmaceutically-acceptable excipient.

18. A method for treating a pulmonary disease in a subject, comprising:
administrating an effective amount of the pharmaceutical composition of claim 1 to the subject in need thereof.

19. A method for treating a pulmonary disease in a subject, comprising:
administrating an effective amount of the pharmaceutical composition of claim 2 to the subject in need thereof.

20. A method for treating a pulmonary disease in a subject, comprising:
administrating an effective amount of the pharmaceutical composition of claim 3 to the subject in need thereof.
